(19) United States
(12) Patent Application Publication
(10) Pub. No.: US 2024/0197702 A1
(43) Pub. Date: Jun. 20, 2024

(54) ANTISEPTIC-ANTIBIOTIC GUMBOS EFFECTIVE AGAINST GRAM-NEGATIVE PATHOGENS

(71) Applicant: Board of Supervisors of Lousiana State University and Agricultural and Mechanical College, Baton Rouge, LA (US)

(72) Inventors: Isiah Manuel WARNER, Baton Rouge, LA (US); Jeffrey Andre HOBDEN, Baton Rouge, LA (US); Kelsey Marie LOPEZ, Baton Rouge, LA (US)

(21) Appl. No.: 17/909,869

(22) PCT Filed: Mar. 10, 2021
(86) PCT No.: PCT/US2021/021608

§ 371 Date: Sep. 7, 2022

Related U.S. Application Data
(60) Provisional application No. 62/987,462, filed on Mar. 10, 2020.

(51) Int. Cl. A61K 31/444; A61K 31/43; A61P 31/04

(52) U.S. Cl. CPC A61K 31/444; A61K 31/43; A61P 31/04

(57) ABSTRACT

Ion-pairs known as GUMBOS (group of uniform materials based on organic salts) from antiseptics (chlorhexidine and octenidine) and the beta-lactam antibiotic, ceftriaxone. The antimicrobial efficacy of these GUMBOS and unreacted stoichiometric equivalent mixtures were compared to ceftriaxone and azithromycin alone. On a molar basis, GUMBOS were equivalent to ceftriaxone and 10× more effective in killing N. gonorrhoeae than azithromycin. They were more than 100× more effective than either antibiotic in killing CRE. A strategy involving the electrostatic interaction between a common antiseptic and a discontinued antibiotic (octenidine and carbenicillin) was also evaluated as a treatment for gonorrhoea. Octenidine/carbenicillin is a novel group of uniform materials based on organic salts (GUMBOS) with inherent in vitro antibacterial activity that comes from its parent antiseptic and antibacterial ions, octenidine and carbenicillin, respectively.

### CROSS-REFERENCE TO RELATED APPLICATION

This application is a National Stage of International Application No. PCT/US2021/021608, filed Mar. 10, 2021, titled “ ANTISEPTIC-ANTIBIOTIC GUMBOS EFFECTIVE AGAINST GRAM-NEGATIVE PATHOGENS” which claims benefit of U.S. Provisional Application No. 62/987,462, filed Mar. 10, 2020, which is hereby incorporated herein by reference in its entirety.

### STATEMENT ON FUNDING PROVIDED BY THE U.S. GOVERNMENT

This invention was made with Government support under contract NNX16AQ93A awarded by NASA and contract CHE-1508726 awarded by the National Science foundation. The Government has certain rights in the invention.

### TECHNICAL FIELD

The present disclosure is generally related to methods and compositions for enhancing the anti-bacterial activity of antibiotics to overcome bacterial antibiotic resistance.

### BACKGROUND

The sexually transmitted infection, gonorrhea, is a global public health threat with the Centers for Disease Control and Prevention (CDC) and World Health Organization (WHO) estimating that more than 820,000 Americans and 78 million people worldwide are infected annually. Gonorrhea affects human superficial mucosal surfaces and is a major cause of pelvic inflammatory disease in the United States. Gonorrhea is easily treated with antibiotics; however, a cause for great concern is that Neisseria gonorrhoeae, the Gram-negative etiological agent of gonorrhea, has shown a limitless capacity for increased resistance to antibiotic treatments. It has become resistant to all antibiotics, except third generation cephalosporins, elevating its status to “superbug”. In 2019, the CDC named N. gonorrhoeae an urgent antibacterial-resistant threat. Since the 1980s, doctors have had to abandon one therapy after another-penicillin, tetracycline, ciprofloxacin, and most recently cefixime. This resistance is due to the ability of N. gonorrhoeae to mutate through two means: chromosomal and plasmid-mediated mutation. One such mutation has led to the production of beta (β)-lactamases, enzymes that provide resistance to antibiotics by breaking the antibiotics' structure rendering it non-effective, which was one of the causes for penicillin resistance. The current treatment for gonorrhea infections, as recommended by the CDC, is now dual antibiotic therapy of ceftriaxone (250 mg intramuscularly) and azithromycin (1 g orally). However, the current antibiotic regimen is being threatened as globally emerging and increasingly resistant strains are developing. In the United States, only 0.2% of isolates had elevated ceftriaxone minimum inhibitory concentrations (MICs) whereas azithromycin had a much larger increase of isolates with elevated MICs. From 2013-2014, isolates with elevated azithromycin MICs had a sharp increase from 0.6% to 2.5% and during 2014-2018, the percentage increased from 2.5% to 4.6%. While few isolates in the United States have exhibited decreased susceptibility to ceftriaxone, WHO has reported an increase in resistant N. gonorrhoeae cases in more than 50 countries. Decreased susceptibility of N. gonorrhoeae to cephalosporins is expected to spread and the threat of returning to the pre-antibiotic era of untreatable infectious disease is very real. This would significantly complicate the ability of providers to treat this bacterium as few antibiotic options are available or in development.

In response to this alarming crisis, a strategy was developed for producing antimicrobial agents as GUMBOS (group of uniform materials based on organic salts) through metathesis reactions. This strategy aims to target N. gonorrhoeae using ceftriaxone-based GUMBOS. GUMBOS are tunable, solid phase organic salts that traditionally use ionic liquid counter-ions but have a defined melting point range from 25-250° C. whereas ionic liquids have melting points below 100° C. By coupling functional cations and/or functional anions, this approach could lead to many applications, including antimicrobial chemotherapeutics. As most antiseptics and antibiotics are in salt forms, antimicrobial GUMBOS can be prepared via ion-exchange reactions. GUMBOS fabricated from such antiseptics and antibiotics have been shown to have lower toxicity and higher efficacy than their constituent parts.

### SUMMARY

The disclosure provides compositions and methods of making and use of GUMBOS (group of uniform materials based on organic salts) compositions that combine an antiseptic and an antibiotic and which are synergistically effective in decreasing the proliferation or viability of a bacterial strain. One aspect of the disclosure encompasses embodiments of a composition comprising an antiseptic in ionic association with an antibiotic as an ion-pair solid-phase organic salt, wherein the antiseptic and the antibiotic can synergistically interact whereby the composition has a Minimal Inhibitory Concentration (MIC) against a sensitive bacterial species that is less than the sum of the MICs of the antiseptic and the antibiotic individually.

In some embodiments of this aspect of the disclosure, the antiseptic can be effective in modulating the proliferation or viability of a Gram-negative strain of a bacterial species when in contact with said strain.

In some embodiments of this aspect of the disclosure, the Gram-negative strain can be a strain of Neisseria gonorrhoeae or a carbapenem-resistant Enterobacteriaceae.

In some embodiments of this aspect of the disclosure, the antiseptic can be effective in modulating the proliferation or viability of a strain of Neisseria gonorrhoeae or a carbapenem-resistant Enterobacteriaceae when in contact with said strain.

In some embodiments of this aspect of the disclosure, the antibiotic can be effective in modulating the proliferation or viability of a carbapenem-resistant bacterial strain.

In some embodiments of this aspect of the disclosure, the antiseptic can be chlorhexidine or octenidine.

In some embodiments of this aspect of the disclosure, the antibiotic can be a β-lactam.

In some embodiments of this aspect of the disclosure, the β-lactam can be ceftriaxone or carbenicillin.

In some embodiments of this aspect of the disclosure, the composition can comprise octenidine and ceftriaxone.

In some embodiments of this aspect of the disclosure, the composition can comprise chlorhexidine and ceftriaxone.

In some embodiments of this aspect of the disclosure, the composition can comprise octenidine and carbenicillin.

In some embodiments of this aspect of the disclosure, the antiseptic and the antibiotic of the antiseptic-antibiotic ionic-pair solid-phase organic salt can be in a stoichiometric ratio of 2:1, 1:1, or 1:2.

In some embodiments of this aspect of the disclosure, the composition can further comprise a pharmaceutical carrier.

Another aspect of the disclosure encompasses embodiments of a pharmaceutical composition comprising a composition of any of claims 1-10 and a pharmaceutical carrier.

In some embodiments of this aspect of the disclosure, the pharmaceutical composition can be formulated for delivery to a subject human or animal intravascularly, or directly to a tissue of the subject.

In some embodiments of this aspect of the disclosure, the pharmaceutical composition can be formulated for delivery to a tissue of the oropharyngeal region of the subject human or animal intravascularly.

In some embodiments of this aspect of the disclosure, the pharmaceutical composition can be formulated for delivery to the tissues of a cavity of a subject human or animal.

Yet another aspect of the disclosure encompasses embodiments of a method of generating a group of uniform materials based on organic salts (GUMBOs) comprising mixing a salt of an antibiotic and a salt of either chlorhexidine or octenidine in water for an extended period, thereby generating an antiseptic-antibiotic ion-pair solid-phase organic salt.

In some embodiments of this aspect of the disclosure, the chlorhexidine salt can be chlorhexidine diacetate salt.

In some embodiments of this aspect of the disclosure, the octenidine salt can be octenidine dihydrochloride.

In some embodiments of this aspect of the disclosure, the antibiotic can be a β-lactam.

In some embodiments of this aspect of the disclosure, the β-lactam can be ceftriaxone or carbenicillin.

Another aspect of the disclosure encompasses embodiments of a method of reducing the proliferation or viability of a bacterial strain comprising contacting a population of the bacterial strain or strains with an antiseptic-antibiotic ion-pair solid-phase organic salt.

In embodiments of this aspect of the disclosure, the antiseptic is effective in modulating the proliferation or viability of a strain of Neisseria gonorrhoeae or a carbapenem-resistant Enterobacteriaceae.

In some embodiments of this aspect of the disclosure, the antiseptic can be chlorhexidine or octenidine.

In some embodiments of this aspect of the disclosure, the antibiotic can be a β-lactam.

In some embodiments of this aspect of the disclosure, the β-lactam can be ceftriaxone or carbenicillin.

In some embodiments of this aspect of the disclosure, the composition can comprise octenidine and ceftriaxone.

In some embodiments of this aspect of the disclosure, the composition can comprise chlorhexidine and ceftriaxone.

In some embodiments of this aspect of the disclosure, the composition can comprise octenidine and carbenicillin.

In some embodiments of this aspect of the disclosure, the antiseptic and the antibiotic of the antiseptic-antibiotic ionic-pair solid-phase organic salt can be in a stoichiometric ratio of 2:1, 1:1, or 1:2.

In some embodiments of this aspect of the disclosure, the composition can further comprise a pharmaceutical carrier.

In some embodiments of this aspect of the disclosure, the composition can be delivered to a subject human or animal having an infection of a gram-negative bacterial strain.

In some embodiments of this aspect of the disclosure, the infection can be of a Neisseria gonorrhoeae or a carbapenem-resistant Enterobacteriaceae.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates a reaction scheme for synthesis of ceftriaxone-based GUMBOS.

FIG. 2 illustrates a reaction scheme for synthesis of carbapenem-based GUMBOS.

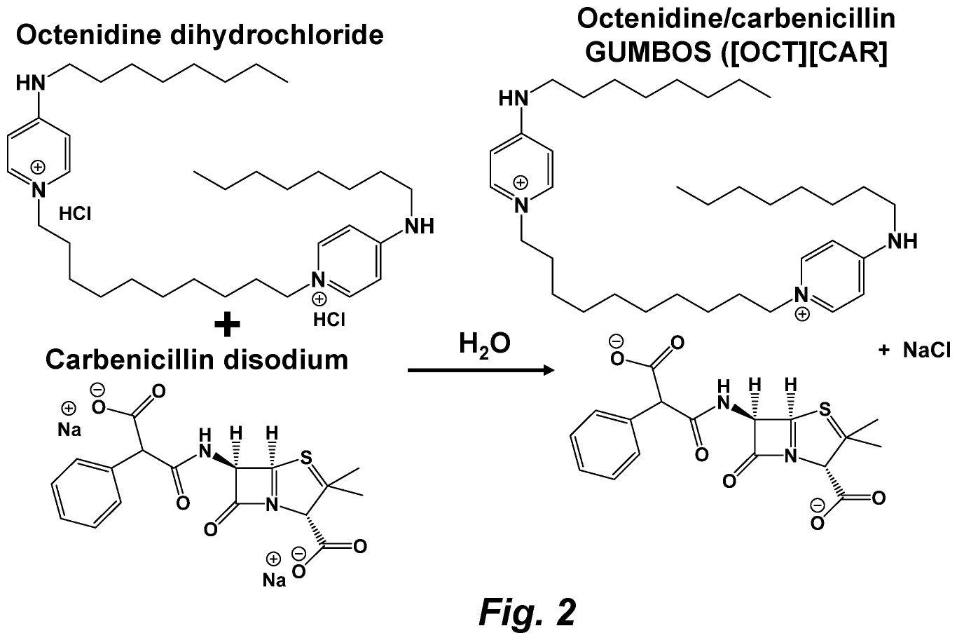

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions

The term “GUMBOS” as used herein refers to a group of solid phase organic salts, typically using ionic liquid counter-ions; however, the melting point range of GUMBOS has been extended beyond that of ionic liquids, to the range of 25-250° C. In contrast, the melting point for ionic liquids lies below 100° C. These compounds have uniquely tunable properties that can be incorporated into the salt via careful selection of counter-ions. GUMBOS are stable and relatively non-toxic as compared with their constituent compounds. Antimicrobial GUMBOS are typically as effective as or better than conventional antibiotic therapy against multi-antibiotic-resistant bacteria. Antimicrobial GUMBOS are simple and inexpensive to synthesize from existing, well-known compounds such as antiseptics and antibiotics in their salt forms.

Numerical ranges recited herein by endpoints include all numbers and fractions subsumed within that range (e.g. 1 to 5 includes 1, 1, 5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a”, “an,” and “the” include plural referents unless the content clearly dictates otherwise. Thus, for example, reference to a composition comprising “a compound” includes a mixture of two or more compounds.

The terms “administering” and “administration” as used herein refer to a process by which a therapeutically effective amount of a compound of the disclosure or compositions contemplated herein are delivered to a subject for prevention and/or treatment purposes. Compositions are administered in accordance with good medical practices considering the subject's clinical condition, the site and method of administration, dosage, patient age, sex, body weight, and other factors known to physicians.

The terms “co-administration” of “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “treating” as used herein refers to reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a compound or composition of the present disclosure to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above. The purpose of prevention and intervention is to combat the disease, condition, or disorder and includes the administration of an active compound to prevent or delay the onset of the symptoms or complications, or alleviating the symptoms or complications, or eliminating the disease, condition, or disorder.

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

The term “therapeutically effective amount” relates to the amount or dose of an active compound of the disclosure or composition comprising the same, that will lead to one or more desired effects, in particular, one or more therapeutic effects or beneficial pharmacokinetic profiles. A therapeutically effective amount of a substance can vary according to factors such as the disease state, age, sex, and weight of the subject, and the ability of the substance to elicit a desired response in the subject. A dosage regimen may be adjusted to provide the optimum therapeutic response or pharmacokinetic profile. For example, several divided doses may be administered daily or the dose may be proportionally reduced as indicated by the exigencies of the therapeutic situation.

The term “beneficial pharmacokinetic profile” refers to amounts or doses of a compound of the disclosure that provide levels of the compound or a required dose resulting in therapeutic effects in the prevention, treatment, or control of symptoms of a disease disclosed herein. The term “sustained pharmacokinetic profile” as used herein refers to a length of time efficacious levels of a biologically active compound of the disclosure is in its environment of use. A sustained pharmacokinetic profile can be such that a single or twice daily administration adequately prevents, treats, or controls symptoms of a disease disclosed herein. A beneficial pharmacokinetic profile may, but is not limited to, providing therapeutically effective amounts of the compound of the disclosure in the subject for about 12 to about 48 h, 12 h to about 36 h, or 12 h to about 24 h.

The term “therapeutic effect” as used herein refers to an effect of a composition of the disclosure, in particular a formulation or dosage form, or method disclosed herein. A therapeutic effect may be a sustained therapeutic effect that correlates with a continuous concentration of a compound of the disclosure over a dosing period, in particular a sustained dosing period. A therapeutic effect may be a statistically significant effect in terms of statistical analysis of an effect of a compound of the disclosure versus the effects without the compound.

“Statistically significant” or “significantly different” effects or levels may represent levels that are higher or lower than a standard. In aspects of the disclosure, the difference may be 1.5, 2, 3, 4, 5, 6, 7, 8, 9, 10, 15, 20, 25 or 50 times higher or lower compared with the effect obtained without a compound of the disclosure.

The term “pharmaceutically acceptable carrier, excipient, or vehicle” as used herein refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

The compounds of the disclosure may also include “pharmaceutically acceptable salt(s)”. By pharmaceutically acceptable salts is meant those salts thatwhich are suitable for use in contact with the tissues of a subject or patient without undue toxicity, irritation, allergic response and the like, and are commensurate with a reasonable benefit/risk ratio. Pharmaceutically acceptable salts are described for example, in S. M, Berge, at al., J. Pharmaceutical Sciences, 1977, 66:1. Suitable salts include salts that may be formed where acidic protons in the compounds are capable of reacting with inorganic or organic bases. Suitable inorganic salts include those formed with alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid). When there are two acidic groups present, a pharmaceutically acceptable salt may be a mono-acid-mono-salt or a di-salt; and similarly where there are more than two acidic groups present, some or all of such groups can be salified.

A compound of the disclosure includes crystalline forms which may exist as polymorphs. Solvates of the compounds formed with water or common organic solvents are also intended to be encompassed within the term. In addition, hydrate forms of the compounds and their salts are encompassed within this disclosure. Further prodrugs of compounds of the disclosure are encompassed within the term.

The term “solvate” means a physical association of a compound with one or more solvent molecules or a complex of variable stoichiometry formed by a solute (for example, a compound of the disclosure) and a solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological, activity of the solute. Solvates encompass both solution-phase and isolatable solvates. Representative solvates include hydrates, ethanolates, methanolates, and the like. Dehydrate, co-crystals, anhydrous, or amorphous forms of the compounds of the disclosure are also included. The term “hydrate” means a solvate wherein the solvent molecule(s) is/are H₂O, including, mono-, di-, and various poly-hydrates thereof. Solvates can be formed using various methods known in the art.

Crystalline compounds of the disclosure can be in the form of a free base, a salt, or a co-crystal. Free base compounds can be crystallized in the presence of an appropriate solvent in order to form a solvate. Acid salt compounds of the disclosure (e.g. HCl, HBr, benzoic acid) can also be used in the preparation of solvates. For example, solvates can be formed by the use of acetic acid or ethyl acetate. The solvate molecules can form crystal structures via hydrogen bonding, van der Waals forces, or dispersion forces, or a combination of any two or all three forces.

The amount of solvent used to make solvates can be determined by routine testing. For example, a monohydrate of a compound of the disclosure would have about 1 equivalent of solvent (H₂O) for each equivalent of a compound of the disclosure. However, more or less solvent may be used depending on the choice of solvate desired.

Compounds of the disclosure may be amorphous or may have different crystalline polymorphs, possibly existing in different salvation or hydration states. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

A compound of the disclosure may be pure or substantially pure. As used herein, the term “pure” in general means better than 90%, 92%, 93%, 94%, 95%, 96%, 97%, 98% or 99% pure, and “substantially pure” means a compound synthesized such that the compound, as made or as available for consideration into a composition or therapeutic dosage described herein, has only those impurities that cannot readily nor reasonably be removed by conventional purification processes.

A compound of the disclosure includes derivatives. As used herein the term “derivative” of a compound of the disclosure refers to a chemically modified compound wherein the chemical modification takes place either at a functional group or ring of the compound. Non-limiting examples of derivatives of compounds of the disclosure may include N-acetyl, N-methyl, N-hydroxy groups at any of the available nitrogens in the compound.

A compound of the disclosure may include a carrier. Suitable carriers include a polymer, carbohydrate, or a peptide.

Therapeutic efficacy and toxicity of compounds, compositions and methods of the disclosure may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions that exhibit large therapeutic indices are preferred. By way of example, one or more of the therapeutic effects can be demonstrated in a subject or disease model by the screening methods of the disclosure.

The disclosure provides dosage forms, formulations, and methods that provide advantages and/or beneficial pharmacokinetic profiles, more particularly sustained pharmacokinetic profiles. A compound of the disclosure can be utilized in dosage forms in pure or substantially pure form, in the form of its pharmaceutically acceptable salts, and also in other forms including anhydrous or hydrated forms.

A beneficial pharmacokinetic profile may be obtained by administering a formulation or dosage form suitable for once, twice a day, or three times a day, or more administration comprising one or more compound of the disclosure present in an amount sufficient to provide the required concentration or dose of the compound to an environment of use to treat a disease disclosed herein, in particular a cancer.

Embodiments of the disclosure relate to a dosage form comprising one or more compound of the disclosure that can provide peak plasma concentrations of the compound of between about 0.001 to 2 mg/ml, 0001 to 1 mg/ml, 0.0002 to 2 mg/ml, 0.005 to 2 mg/ml, 001 to 2 mg/ml, 0.05 to 2 mg/ml, 0.001 to 0.5 mg/ml, 0.002 to 1 mg/ml, 0.005 to 1 mg/ml, 0.01 to 1 mg/ml, 005 to 1 mg/ml, or 0.1 to 1 mg/ml. The disclosure also provides a formulation or dosage form comprising one or more compound of the disclosure that provides an elimination t_{1/2} of 0.5 to 20 h, 0.5 to 15 h, 0.5 to 10 h, 0.5 to 6 h, 1 to 20 h, 1 to 15 h, 1 to 10 h, or 1 to 6 h.

A subject may be treated with a compound of the disclosure or composition or unit dosage thereof on substantially any desired schedule. They may be administered one or more times per day, in particular 1 or 2 times per day, once per week, once a month or continuously. However, a subject may be treated less frequently, such as every other day or once a week, or more frequently. A compound or composition may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously.

A beneficial pharmacokinetic profile can be obtained by the administration of a formulation or dosage form suitable for once, twice, or three times a day administration, preferably twice a day administration comprising one or more compound of the disclosure present in an amount sufficient to provide the requited dose of the compound. The required dose of a compound of the disclosure administered once twice, three times or more daily is about 0.01 to 3000 mg/kg, 0.01 to 2000 mg/kg, 0.5 to 2000 mg/kg, about 0.5 to 1000 mg/kg, 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 50 mg/kg, 0.1 to 20 mg/kg, 0.1 to 10 mg/kg, 0.1 to 6 mg/kg, 0.1 to 5 mg/kg, 0.1 to 3 mg/kg, 0.1 to 2 mg/kg, 0.1 to 1 mg/kg, 1 to 1000 mg/kg, 1 to 500 mg/kg, 1 to 400 mg/kg, 1 to 300 mg/kg, 1 to 200 mg/kg, 1 to 100 mg/kg, 1 to 50 mg/kg, 1 to 20 mg/kg, 1 to 10 mg/kg, 1 to 6 mg/kg, 1 to 5 mg/kg, or 1 to 3 mg/kg, or 1 to 2.5 mg/kg, or less than or about 10 mg/kg, 5 mg/kg, 2.5 mg/kg, 1 mg/kg, or 0.5 mg/kg twice daily or less

The disclosure also contemplates a formulation or dosage form comprising amounts of one or more compound of the disclosure that results in therapeutically effective amounts of the compound over a dosing period, in particular a 24 h dosing period. The therapeutically effective amounts of a compound of the disclosure are between about 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 75 mg/kg, 0.1 to 50 mg/kg, 0.1 to 25 mg/kg, 0.1 to 20 mg/kg, 0.1 to 15 mg/kg, 0.1 to 10 mg/kg, 0.1 to 9 mg/kg, 0.1 to 8 mg/kg, 0.1 to 7 mg/kg, 0.1 to 6 mg/kg, 0.1 to 5 mg/kg, 0.1 to 4 mg/kg, 0.1 to 3 mg/kg, 0.1 to 2 mg/kg, or 0.1 to 1 mg/kg.

A medicament or treatment of the disclosure may comprise a unit dosage of at least one compound of the disclosure to provide therapeutic effects. A “unit dosage” or “dosage unit” refers to a unitary, i.e. a single dose, which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A formulation or dosage form of the disclosure may be an immediate release dosage form or a non-immediate release delivery system, including without limitation a delayed-release or sustained-release dosage form.

The disclosure provides a sustained-release dosage form of a compound of the disclosure which advantageously achieves a more sustained drug plasma while mitigating or eliminating drug concentration spikes by providing a substantially steady release of the compound over time. A substantially constant plasma concentration preferably correlates with one or more therapeutic effects disclosed herein. In embodiments, the sustained-release dosage form is for oral administration.

A composition, in particular a dosage form or formulation, may be in any form suitable for administration to a subject, including without limitation, a form suitable for oral, parenteral, intravenous (bolus or infusion), intraperitoneal, subcutaneous, or intraffinsculas administration. A dosage form or formulation may be a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder.

A dosage form or formulation can be an oral dosage form or formulation such as tablets, caplets, soft and hard gelatin capsules, pills, powders, granules, elixirs, tinctures, suspensions, syrups, and emulsions. The dosage form or formulation can be a parenteral dosage form such as an active substance in a sterile aqueous or non-aqueous solvent, such as water, isotonic saline, isotonic glucose solution, buffer solution, or other solvents conveniently used for parenteral administration.

A composition of the disclosure can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the disclosure, e.g. encapsulation in liposomes, microparticles, microcapsules, and the like.

Formulations for parenteral administration may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

A composition of the disclosure may be sterilized by, for example, filtration through a bacteria retaining filter, addition of sterilizing agents to the composition, irradiation of the composition, or heating the composition. Alternatively, the compounds or compositions of the present disclosure may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the disclosure, such labeling would include amount, frequency, and method of administration.

### Discussion

Neisseria gonorrhoeae is evolving to become resistant to the currently recommended antibiotics ceftriaxone and azithromycin. New antibacterial agents resistant to inactivation by N. gonorrhoeae are urgently needed. In this study, we fashioned ion-pairs known as GUMBOS (group of uniform materials based on organic salts) from antiseptics (chlorhexidine and octenidine) and the beta-lactam antibiotic, ceftriaxone. The antimicrobial efficacy of these GUMBOS and unreacted stoichiometric equivalent mixtures were compared to ceftriaxone and azithromycin alone. Susceptibility testing on N. gonorrhoeae ATCC 49226 and four clinical isolates of N. gonorrhoeae was performed using micro-broth dilution. To further demonstrate the value of GUMBOS, we also tested three clinical isolates of carbapenem-resistant Enterobacteriaceae (CRE). Cytotoxicities of GUMBOS, unreacted mixtures, and antiseptics were evaluated using an MTT assay. On a molar basis, GUMBOS were equivalent to ceftriaxone and 10× more effective in killing N. gonorrhoeae than azithromycin. They were more than 100× more effective than either antibiotic in killing CRE. Finally, GUMBOS have a safety profile similar to their antiseptic components, suggesting that these unique compounds will be clinically useful as antibacterial agents.

The present disclosure encompasses the fabrication and characterization of GUMBOS from common antiseptics, chlorhexidine diacetate and octenidine dihydrochloride, and ceftriaxone disodium salt and their efficacy in killing N. gonorrhoeae. The efficacy of these GUMBOS against members of carbapenem-resistant Enterobacteriaceae (CRE), a family of multi-drug resistant Gram-negative bacteria that pose a global threat to human health, is also demonstrated.

The present disclosure encompasses methods of reducing oropharyngeal gonorrhea, as drug-resistant N. gonorrhoeae isolates from the oropharynx have emerged, most likely because there is poor drug penetration into pharyngeal tissue.³ While current studies may have limitations, it has been recently demonstrated that gonococci could possibly be transmitted person to person strictly from ‘deep kissing’. N. gonorrhoeae can also persist in the oropharynx as most cases are asymptomatic or are misdiagnosed as other kinds of pharyngitis. The oropharynx is thought to act as a ‘silent reservoir’, in which the gonococcus acquires its resistance determinants by horizontal gene transfer from commensal Neisseria species and other bacterial species in asymptomatic individuals. For this reason, decolonization of these individuals might prevent the emergence of MDR gonorrhea.

Currently, the recommended treatment is a dual regimen of ceftriaxone administered intramuscularly and azithromycin orally. Although these antibiotics remain generally effective, resistance rates are rising globally. Antibiotic-resistant oropharyngeal gonorrhea threatens to become a global crisis as conventional antibiotic therapy does not always reliably clear the pathogen from the throat. In 2010, a Swedish heterosexual man who presented with oropharyngeal gonorrhea required several rounds of ceftriaxone with increasing dosage. In 2017, a heterosexual man from the UK acquired drug-resistant oropharyngeal gonorrhea in Southeast Asia. The infection did not respond to a high dosage of ceftriaxone (1 g) and spectinomycin, which indicates high-level resistance. The infection was cleared only after IV administration of ertapenem, an antibiotic belonging to the potent carbapenem class of b-lactams that are often agents of ‘last resort’

A possible solution for oropharyngeal gonorrhea could be use of antiseptics, as they are unlikely to induce resistance. Antiseptics have been explored as treatments for acute gonorrhea in the past and antiseptic mouthwashes have been shown to have an antimicrobial effect on oropharyngeal gonorrhea. One such antiseptic, octenidine dihydrochloride, exhibits a broad spectrum of antimicrobial activity against Gram-negative and Gram-positive bacteria. This antiseptic has shown significant efficacy in periodontology and is even sold as a commercially available mouth-wash under the name OcteniseptVR. It has also been approved for use on skin, mucosal membranes and as wound antisepsis. The present disclosure provides a strategy for reducing gonorrhea infections by developing an antiseptic-based compound that can be used as alternative therapy for gonorrhea, specifically oropharyngeal gonorrhea.

Bringing new antibiotics to market requires not only years of research and development but can also cost billions of dollars. In response to this global crisis, the literature supports the use of GUMBOS (group of uniform materials based on organic salts) as antimicrobial agents.

Antibacterial activities for octenidine dihydrochloride, disodium carbenicillin, octenidine/carbenicillin and stoichiometrically equivalent 1:1 octenidine dihydrochloride to disodium carbenicillin were assessed using the Kirby-Bauer disc diffusion assay for N. gonorrhoeae (ATCC 49226) and three clinical isolates. Predictive permeability using the Parallel Artificial Membrane Permeability Assay and cytotoxicity against HeLa cells was also evaluated.

Additive in vitro antibacterial activities against N. gonorrhoeae were observed, which suggests octenidine/carbenicillin could be a useful agent in reducing N. gonorrhoeae transmission and minimizing gonorrhea infections. Octenidine/carbenicillin also exhibited bioequivalence to azithromycin and doxycycline, two currently prescribed antibiotics. Likewise, octenidine/carbenicillin had improved predicted permeability compared with octenidine dihydrochloride.

Antimicrobial GUMBOS synthesized in this study could be used as an adjunctive treatment approach to current drug therapies for oropharyngeal gonorrhea infection control and prevention.

As the threat of antibiotic resistance in N. gonorrhoeae increases and oropharyngeal cases become more difficult to treat, alternative therapies are greatly needed. This study suggests that octenidine/ carbenicillin GUMBOS may be a viable alternative therapy for prevention and minimization of N. gonorrhoeae transmission. GUMBOS were easily synthesized using ion-exchange reactions in deionized water. Octenidine/carbenicillin was found to be bioequivalent to azithromycin and doxycycline, as determined by Kirby-Bauer disc diffusion assays. Moreover, octenidine/carbenicillin exhibited higher efficacy than the constituent parent compounds and unreacted mixtures. Cytotoxicity results showed that octenidine/carbenicillin was also non-toxic towards cervical cells. This approach of fashioning antimicrobial agents into GUMBOS may offer an alternative approach to current drug therapies for gonorrhea and have further implications for topical prevention strategies.

One aspect of the disclosure encompasses embodiments of a composition comprising an antiseptic in ionic association with an antibiotic as an ion-pair solid-phase organic salt, wherein the antiseptic and the antibiotic can synergistically interact whereby the composition has a Minimal Inhibitory Concentration (MIC) against a sensitive bacterial species that is less than the sum of the MICs of the antiseptic and the antibiotic individually.

In some embodiments of this aspect of the disclosure, the antiseptic can be effective in modulating the proliferation or viability of a gram-negative strain of a bacterial species when in contact with said strain.

In some embodiments of this aspect of the disclosure, the gram-negative strain can be a strain of Neisseria gonorrhoeae or a carbapenem-resistant Enterobacteriaceae.

In some embodiments of this aspect of the disclosure, the antiseptic can be effective in modulating the proliferation or viability of a strain of Neisseria gonorrhoeae or a carbapenem-resistant Enterobacteriaceae when in contact with said strain.

In some embodiments of this aspect of the disclosure, the antibiotic can be effective in modulating the proliferation or viability of a carbapenem-resistant bacterial strain.

In some embodiments of this aspect of the disclosure, the antiseptic can be or octenidine.

In some embodiments of this aspect of the disclosure, the antibiotic can be chlorhexidine a β-lactam.

In some embodiments of this aspect of the disclosure, the β-lactam can be ceftriaxone or carbenicillin.

In some embodiments of this aspect of the disclosure, the composition can comprise octenidine and ceftriaxone.

In some embodiments of this aspect of the disclosure, the composition can comprise chlorhexidine and ceftriaxone.

In some embodiments of this aspect of the disclosure, the composition can comprise octenidine and carbenicillin.

In some embodiments of this aspect of the disclosure, the antiseptic and the antibiotic of the antiseptic-antibiotic ionic-pair solid-phase organic salt can be in a stoichiometric ratio of 2:1, 1:1, or 1:2 depending on the respective charges of the antiseptic and antibiotic.

In some embodiments of this aspect of the disclosure, the composition can further comprise a pharmaceutical carrier.

Another aspect of the disclosure encompasses embodiments of a pharmaceutical composition comprising a composition of any of claims 1-10 and a pharmaceutical carrier.

In some embodiments of this aspect of the disclosure, the pharmaceutical composition can be formulated for delivery to a subject human or animal intravascularly, or directly to a tissue of the subject.

In some embodiments of this aspect of the disclosure, the pharmaceutical composition can be formulated for delivery to a tissue of the oropharyngeal region of the subject human or animal intravascularly.

In some embodiments of this aspect of the disclosure, the pharmaceutical composition can be formulated for delivery to the tissues of a cavity of a subject human or animal.

Yet another aspect of the disclosure encompasses embodiments of a method of generating a group of uniform materials based on organic salts (GUMBOS) comprising mixing a salt of an antibiotic and a salt of either chlorhexidine or octenidine in water for an extended period, thereby generating an antiseptic-antibiotic ion-pair solid-phase organic salt.

In some embodiments of this aspect of the disclosure, the chlorhexidine salt can be chlorhexidine diacetate salt.

In some embodiments of this aspect of the disclosure, the octenidine salt can be octenidine dihydrochloride.

In some embodiments of this aspect of the disclosure, the antibiotic can be ceftriaxone or a β-lactam.

In some embodiments of this aspect of the disclosure, the β-lactam can be carbenicillin.

Another aspect of the disclosure encompasses embodiments of a method of reducing the proliferation or viability of a bacterial strain comprising contacting a population of the bacterial strain or strains with an antiseptic-antibiotic ion-pair solid-phase organic salt.

In embodiments of this aspect of the disclosure, the antiseptic is effective in modulating the proliferation or viability of a strain of Neisseria gonorrhoeae or a carbapenem-resistant Enterobacteriaceae.

In some embodiments of this aspect of the disclosure, the antiseptic can be chlorhexidine or octenidine.

In some embodiments of this aspect of the disclosure, the antibiotic can be a β-lactam.

In some embodiments of this aspect of the disclosure, the β-lactam can be ceftriaxone or carbenicillin.

In some embodiments of this aspect of the disclosure, the composition can comprise octenidine and ceftriaxone.

In some embodiments of this aspect of the disclosure, the composition can comprise chlorhexidine and ceftriaxone.

In some embodiments of this aspect of the disclosure, the composition can comprise octenidine and carbenicillin.

In some embodiments of this aspect of the disclosure, the antiseptic and the antibiotic of the antiseptic-antibiotic ionic-pair solid-phase organic salt can be in a stoichiometric ratio of 2:1, 1:1, or 1:2.

In some embodiments of this aspect of the disclosure, the composition can further comprise a pharmaceutical carrier.

In some embodiments of this aspect of the disclosure, the composition can be delivered to a subject human or animal having an infection of a gram-negative bacterial strain.

In some embodiments of this aspect of the disclosure, the infection can be of a Neisseria gonorrhoeae or a carbapenem-resistant Enterobacteriaceae.

While embodiments of the present disclosure are described in connection with the examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

### Example 1

Materials: Ceftriaxone disodium salt, octenidine hydrochloride, and azithromycin were purchased from TCI. Chlorhexidine diacetate was purchased from Acros Organics. A cell viability MTT (3-[4,5-Dimethylthiazol-2-yl]-2,5-diphenyltetrazolium bromide) assay was purchased from Promega Corporation (Madison, WI). Prepared agar plates (BD BBL Prepared Plate Media: GC II agar with IsoVitalex® Enrichment) and Oxoid® antimicrobial susceptibility discs were purchased from Fisher Scientific (Pittsburg, PA). Cation-adjusted Muller Hinton Brother and reagents for preparing a fully defined, clear, protein-free liquid media as described by Wade and Graver (Fed. Euro. Microbiol. Soc. Microbiol. Letts (2007), 273 (1), 35-37) were purchased from Fisher Scientific (Pittsburgh, PA).

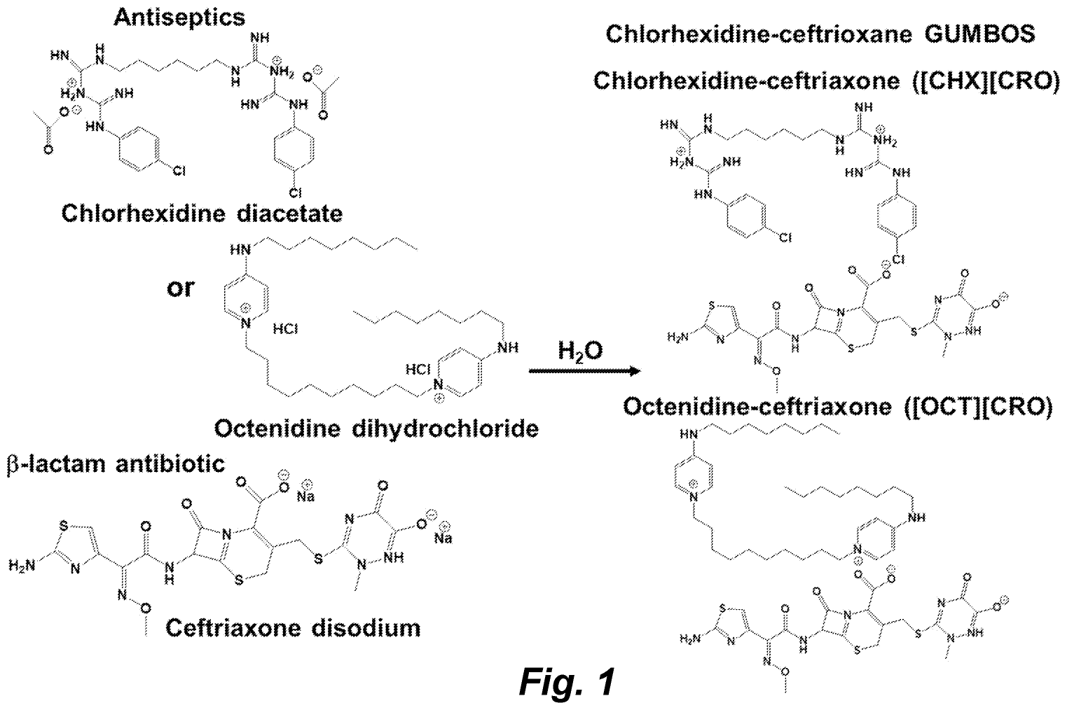

Synthesis of Ceftriaxone-based GUMBOS: Ceftriaxone-based GUMBOS were synthesized by use of ion-exchange reactions in deionized water. This reaction scheme is illustrated in FIG. 1. Briefly, stoichiometrically equivalent amounts of disodium ceftriaxone and antiseptic, chlorhexidine diacetate (CHX 2Ac) and octenidine dihydrochloride (OCT 2HCl), were mixed for two hours at room temperature. The resulting products were washed several times with cold, deionized water and lyophilized overnight. The resulting ceftriaxone-based GUMBOS, chlorhexidine-ceftriaxone ([CHX][CRO]) and octenidine-ceftriaxone ([OCT][CRO]), were characterized using nuclear magnetic resonance (NMR) and mass spectrometry.

Octanol-Water Partition Coefficient: To determine the relative lipophilic strength of GUMBOS, the logarithmic octanol-water partition coefficient (Log P) was measured. It is a ratiometric parameter based on the concentration of therapeutic agent in either phase of the two-phase system (octanol and water) when at equilibrium (Equation 1). Specifically, a shake-flask method was used. To prevent minimal solvent miscibility, an equal amount of water and 1-octanol were mixed together for 24 hours and subsequently allowed to separate. The octanol layer was used to generate a calibration curve from the absorbance of the compound at various concentrations. An equal volume of saturated water was added to a flask of known concentration (Cᵢ) chosen from the calibration curve. This mixture was stirred for a minimum of two hours and left undisturbed to allow the two solvent layers to separate. The absorbance of the therapeutic salt in the octanol layer was determined (Cₒ). Thus, the concentration of therapeutic agent in water was determined using Cᵢ−Cₒ=C_{w}.

$$Log P = \frac{[ Octanol ]}{[ Water ]} \qquad ( 1 )$$

Minimum Inhibitory Concentrations of GUMBOS against N. gonorrhoeae: Clinical isolates of N. gonorrhoeae and N. gonorrhoeae ATCC 49226 were obtained from Louisiana State University Health Sciences Center New Orleans HIV Outpatient Clinic. Minimum inhibitory concentrations (MICs) of GUMBOS, constituent parent drugs, azithromycin, and unreacted mixtures were determined using micro-broth dilution in 96-well plates. Stock solutions of 1 mM were prepared in deionized water (1% DMSO). Stock solutions were diluted to a known working concentration and 100 μL were added to the first row of the plate. For susceptibility testing of N. gonorrhoeae, media was prepared according to methods reported by Wade and Graver (Fed. Euro. Microbiol. Soc. Microbiol. Letts (2007), 273 (1), 35-37). This liquid medium allowed dense growth of N. gonorrhoeae for use in broth dilution tests. Protein-free liquid media (100 μL) were added to each well and two-fold dilutions were performed. The antibiotic containing wells were inoculated with an equal volume of bacterial inoculum matching a 0.5 McFarland standard. Microtiter plates were incubated for 20-24 hours at 37° C. with 5% CO₂ and examined for visible signs of bacterial growth as evidenced by turbidity. The lowest concentration of antibiotic that prevents growth is the MIC.

Minimum Inhibitory Concentrations of GUMBOS against Carbapenem-resistant Enterobacteriaceae: GUMBOS, ceftriaxone, and azithromycin were tested against extended spectrum beta-lactamase (ESBL)-producing gram negative bacteria. Carbapenem-resistant clinical isolates of Escherichia coli, Enterobacter cloacae, and Klebsiella pneumoniae were obtained from Louisiana State University (LSU) Health Sciences Center New Orleans HIV Outpatient Clinic. Minimum inhibitory concentrations were determined as described above using micro-broth dilutions with two minor adjustments, cation-adjusted Mueller Hinton (MH) broth was used instead and stock solutions of test compounds were prepared directly in MH broth.

Cytotoxicity Assay of GUMBOS: Cytotoxicity of GUMBOS, unreacted mixtures, and antiseptics towards healthy cells were evaluated using the MTT assay, a colorimetric dye assay, according to manufacturer's instructions. HeLa (ATCC CL-2) cells were grown in Dulbecco's modified Eagle's medium-reduced serum (DMEM) supplemented with 10% fetal bovine serum and plated at a density of 1×10⁵ cells/mL (10⁴ cells/well) in 96-well plates. Stock solutions of antimicrobial agents in cell medium (1% MeOH) were diluted to a known working concentration, doubly diluted, and transferred to seeded cells. Cells were incubated for 15 minutes at 37° C. in 5% CO₂ atmosphere. Cells treated with only DMEM were used as a negative control. After incubation, 15 μL of MTT were added to each well and cells were further incubated for an hour. Sodium dodecyl sulfate dimethylformamide solution was added after this additional incubation time in order to dissolve the purple formazan crystals. Cell viability was quantified at 570 nm using a microplate spectrophotometer (Eppendorf PlateReader AF2200, Hauppauge, NY). Cell viability as a percentage was determined as the ratio between treated cells and untreated (control) cells taken as 100%.

### Characterization of Ceftriaxone-Based GUMBOS

### [CHX][CRO]

¹H-NMR (400HZ, DMSO-d6) δ 10.61 (s, 2 H), 9.53 (d, J=8 Hz, 1 H), 8.38 (br. s, 1 H), 7.95 (s, 1 H), 7.64 (m, 10 H), 7.29 (d, J=8.8 Hz, 4 H), 7.21 (s, 2 H), 6.74 (s, 1 H), 5.61 (dd, J=8.2 Hz, 1 H), 5.06 (d, J=0.08 Hz, 1 H), 4.36 (d, J=12.4 Hz, 1 H), 4.14 (d, J=12 Hz, 1 H), 3.82 (m, 3 H), 3.06 (s, 4 H), 2.89 (s, 1 H), 2.73 (s, 1 H), 1.89 (s, 1 H), 1.43 (s, 4 H), 1.23 (s, 4H) ¹³C-NMR (125 MHZ, DMSO) δ 168.35, 165.78, 163.05, 162.05, 153.92, 149.07, 142.61, 132.96, 128.24, 122.06, 115.05, 108.95, 61.84, 58.23, 57.49, 34.27, 26.45. HRMS (ESI) m/z calc. for C₁₈H₁₈N₈O₇S₃, [M+H], 555.0544; found 555.0531. C₂₂H₃₀Cl₂N₁₀, [M+H], 505.2105; found 505.2119.

### [OCT][CRO]

¹H-NMR (400Hz, DMSO-d6) δ 9.47 (d, J=8 HZ, 1 H), 9.26 (t, J=5.6 Hz, 2H), 8.27 (d, J=7.6 Hz, 2H), 8.12 (dd, J=7.2 Hz, 2H), 7.18 (s, 2 H), 7.00 (dd, J=7.2 Hz, 2H), 6.88 (dd, J=7.2 Hz, 2H), 6.73 (s, 1H). 5.51 (dd, J=8 Hz, 1H), 4.96 (d, J=4.8 Hz, 1H), 4.31 (d, J=12.4 Hz, 1H), 4.13 (s, 1H), 4.08 (t, J=7.2 Hz, 4H), 3.82 (s, 2H), 3.51 (s, 1H), 3.46 (s, 1 H), 3.38 (s, 3H), 3.25 (dd, J=12.8 Hz, 6H), 1.72 (q, J=7.2 Hz, 4H), 1.55 (m, 4H), 1.27 (m, 33H), 0.85 (t, J=6.8 Hz, 6H) ¹³C-NMR (125 MHZ, DMSO) δ 168.36, 163.66, 163.06, 161.67, 156.65, 155.50, 149.09, 143.48, 142.63, 141.10, 110.64, 108.99, 104.97, 61.81, 58.12, 57.39, 56.69, 31.22, 28.68, 28.65, 27.92, 26.32, 22.08, 13.94. HRMS (ESI) m/z calc. for C₁₈H₁₈N₈O₇S₃[M+H], 555.0544; found, 555.0527. C₃₆H₆₂N₄[M+H], 551.5047; found, 551.50696.

Predictive Lipophilic Behavior: GUMBOS were synthesized in water and are more hydrophobic than their constituent compounds. However, octanol-water partition coefficients were measured for GUMBOS using the shake flask method to further explore the lipophilic properties of these compounds. The computed Log P values for [CHX][CRO] and [OCT][CRO] were 0.2 (±0.1) and 0.3 (±0.2), respectively. The positive Log P values for GUMBOS indicate a greater affinity for the lipid phase. By exchanging the anions on chlorhexidine with ceftriaxone, [CHX][CRO] exhibited slightly higher lipophilicity than chlorhexidine diacetate. According to Hansch et al (1995), Log P for chlorhexidine is 0.08.²⁴ Octenidine dihydrochloride does not permeate through skin, mucosal membranes, or the placental barrier. Thus by anion exchanging the chloride ions for ceftriaxone, the lipophilicity was increased.

Antimicrobial Activities of Ceftriaxone-based GUMBOS: The antimicrobial activities of [CHX][CRO] and [OCT][CRO] were assessed against the individual precursor components, stoichiometrically equivalent mixture, and azithromycin. This is illustrated in Table 1.

TABLE 1
MICs (μM) of CDC recommended antibiotics, GUMBOS,
their unreacted stoichiometric equivalent mixtures,
octenidine dihydrochloride, and chlorhexidine diacetate

| Antimicrobial Agents | ATCC 49226 | Clinical Isolates |
|---|---|---|
| Ceftriaxone | 0.04 | 0.02 |
| Azithromycin | 0.8 | 0.6 ± 0.2 |
| Chlorhexidine − Ceftriaxone | 0.08 | 0.06 ± 0.02 |
| GUMBOS | | |
| Ceftriaxone + Chlorhexidine mixture | 3.1 | 0.12 ± 0.05 |
| Octenidine − Ceftriaxone GUMBOS | 0.08 | 0.09 ± 0.07 |
| Ceftriaxone + Octenidine mixture | 3.1 | 0.1 |
| Octenidine dihydrochloride | 3.1 | 4.7 ± 1.8 |
| Chlorhexidine diacetate | 3.1 | 4.7 ± 1.8 |

Studies were conducted with N. gonorrhoeae ATCC 49226 and four clinical isolate strains obtained through the LSU Health Sciences Center New Orleans HIV Outpatient Clinic. Evaluation of initial antimicrobial studies with N. gonorrhoeae ATCC 49226 suggested that GUMBOS were more effective than their precursor antiseptics, stoichiometric equivalent mixtures, and azithromycin. GUMBOS required concentrations of 10× less than azithromycin, and concentrations of about 4× less than the stoichiometric equivalent mixtures. Ceftriaxone had a half-fold decrease in concentration as compared to GUMBOS. As expected, ceftriaxone was highly effective against the isolate population since clinical isolates obtained in Louisiana, USA are ceftriaxone susceptible. Azithromycin required concentrations of six to nine times greater than GUMBOS for growth inhibition. Stoichiometric mixtures of antiseptic and ceftriaxone were just as effective as GUMBOS which can be attributed to the ceftriaxone component as chlorhexidine diacetate and octenidine dihydrochloride required concentrations of 50-80× times greater than GUMBOS to inhibit growth of N. gonorrhoeae.

Antimicrobial Activities of Ceftriaxone-based GUMBOS against CRE: The unique chemistry of GUMBOS is thought to make them refractive to existing methods of antibiotic resistance such as β-lactamases. Carbapenemases are a group of enzymes capable of hydrolyzing third generation cephalosporins, such as ceftriaxone²⁷ and antibiotics belonging to the carbapenem class of antibiotics. Carbapenems are members of the β-lactam class of antibiotics and are deemed “antibiotics of last resort”. GUMBOS, ceftriaxone, and azithromycin were tested against various strains of CRE (Escherichia coli, Enterobacter cloacae, Klebsiella pneumoniae) to determine if GUMBOS would provide an advantage over current treatment regimes. The MICs of ceftriaxone, azithromycin, and GUMBOS tested against CRE are reported in Table 2.

TABLE 2
MICs (μM) of ceftriaxone (CRO), azithromycin
(AZM), and GUMBOS against CRE.

| Antimicrobial | E. coli | E. Coli | K. | E. |
|---|---|---|---|---|
| Agents | (Isolate 1) | (Isolate 2) | pneumoniae | cloacae |
| CRO | >500 | >500 | >500 | >500 |
| AZM | >500 | 250 | 250 | 250 |
| [CHX][CRO] | 25 | 12.5 | 50 | 50 |
| [OCT][CRO] | 50 | 25 | 25 | 25 |

High concentrations of ceftriaxone and azithromycin were required to inhibit CRE growth whereas much lower concentrations of GUMBOS were needed to inhibit growth. Chlorhexidine ceftriaxone needed concentrations of 5 to 40× less than ceftriaxone and azithromycin in order to inhibit CRE growth, whereas octenidine ceftriaxone needed concentrations of 5 to 20× less than ceftriaxone and azithromycin. This antimicrobial activity could be attributed to the bulkiness of the chlorhexidine and octenidine cations. The bulkiness could sterically hinder attachment of β-lactamases allowing ceftriaxone to remain active. This may impart a new treatment approach for resistant infections when enzymes are used in antibiotic deactivation mechanisms.

Cytotoxicity Assessment: Cytotoxicity of GUMBOS, antiseptics, and unreacted, stoichiometric mixtures were assessed in order to determine in vitro safety. The antiseptics, chlorhexidine diacetate and octenidine dihydrochloride, have been shown to be non-toxic to various cell lines. Cervical cells were used in these studies to determine an overall, systemic toxicity and to evaluate probable use in eradicating gonorrheal infections of the cervix (Table 3).

TABLE 3
Acute cytotoxicity (IC₅₀, μM) of GUMBOS, unreacted
mixtures, and antiseptics for HeLa cells.

| Antimicrobial Agents | 15 min | 60 min |
|---|---|---|
| Chlorhexidine diacetate | 80.78 ± 4.27 | 24.59 ± 0.66 |
| Octenidine dihydrochloride | 55.01 ± 1.29 | 25.87 ± 1.17 |
| [CHX][CRO] | 84.27 ± 5.87 | 45.49 ± 1.42 |
| 1 CHX:1 CRO | 60.39 ± 6.51 | 49.96 ± 1.07 |
| [OCT][CRO] | 66.51 ± 8.24 | 43.31 ± 5.98 |
| 1 OCT:1 CRO | 68.48 ± 4.16 | 22.24 ± 1.76 |

Cell toxicity caused by GUMBOS was less for [CHX][CRO] than [OCT][CRO]. Chlorhexidine-ceftriaxone was significantly less toxic than its unreacted, stoichiometric mixture (p<0.05); however, octenidine-ceftriaxone showed indifferent toxicity to its unreacted, stoichiometric mixture of parent compounds. In comparison to chlorhexidine diacetate, [CHX][CRO] was only slightly less toxic. However, [OCT][CRO] was significantly less toxic than octenidine dihydrochloride.

As a result of isolates with elevated ceftriaxone and azithromycin MICs on the rise, there is an ongoing need for alternative treatments for gonorrhea. Such urgency is paramount since Neisseria gonorrhea has steadily become resistant to all previous treatments. Thus, it is only a matter of time before susceptibility to ceftriaxone disappears. As expected, ceftriaxone remained susceptible against the clinical isolates obtained for these studies. GUMBOS did not prove to be more effective than ceftriaxone, however; ceftriaxone-based GUMBOS proved to be more effective than azithromycin according to disk diffusion and micro-broth dilution studies. Azithromycin resistance is increasing more rapidly than ceftriaxone resistance and alternative therapies need to be explored. By fashioning ceftriaxone into GUMBOS using antiseptics, the unique structure of these ion-pairs is also shown to be much less affected by beta-lactamases than ceftriaxone disodium salt, thereby extending its spectrum of antibacterial activity against N. gonorrhoeae. While stoichiometric equivalent mixtures of antiseptic and ceftriaxone proved to be as effective as GUMBOS, GUMBOS should still be considered as alternative therapy since they are less toxic and much less affected by beta-lactamases than ceftriaxone. It has also been proven by previous studies that by forming ion-pairs from antiseptics and β-lactam antibiotics, the reduced aqueous solubility leads to greater lipophilicity and increased intramolecular interactions Reduced solubility also relates to potentially greater bioavailability. Overall, this approach may offer an alternative strategy to tackling the impending resistance of N. gonorrhoeae towards ceftriaxone and azithromycin.

### Example 2

Reducing Neisseria gonorrhoeae colonies in the oropharynx is a viable solution to minimize the transmission of this bacterium amongst individuals. The present disclosure encompasses embodiments of a method of generating an octenidine/ carbenicillin GUMBO involving the electrostatic interaction between a common antiseptic and a discontinued antibiotic (i.e. octenidine and carbenicillin) that was evaluated as a potential treatment for gonorrhoea. Octenidine/carbenicillin is a novel group of uniform materials based on organic salts (GUMBOS) with inherent in vitro antibacterial activity that comes from its parent antiseptic and antibacterial ions, octenidine and carbenicillin, respectively.

The present disclosure, therefore, encompasses GUMBOS synthesized from octenidine and carbenicillin, a β-lactam antibiotic with high efficacy against various Gram-negative bacteria and with increased thermal and pH stability in solution. However, carbenicillin is no longer administered due to toxicity issues at high concentrations. By ion-exchanging the sodium ions on carbenicillin with octenidine, the toxicity situation was improved. The in vitro antibacterial efficacy of GUMBOS against N. gonorrhoeae ATCC 49226 and clinical isolates of N. gonorrhoeae was evaluated using disc diffusion susceptibility tests. Comparative analyses of octenidine/carbenicillin GUMBOS, their constituent parts, unreacted stoichiometric mixtures and current treatments for gonorrhea confirm the advantageous use of this approach as an alternative therapy against the threat of antibiotic resistance. The toxicities of these GUMBOS were also evaluated.

Materials and methods: Disodium carbenicillin, methanol (MeOH) and DMSO were purchased from Sigma-Aldrich (Milwaukee, WI, USA). Octenidine dihydrochloride was purchased from TCI Chemicals (Japan). The cell viability MTT assay was purchased from Promega Corporation (Madison, WI, USA). Prepared agar plates (BD BBL Prepared Plated Media: GC II Agar with IsoVitalex Enrichment® and Oxoid® antimicrobial susceptibility discs [ceftriaxone (30 μg), azithromycin (15 μg), doxycycline (30 μg) and blank discs; 6 mm] were purchased from Thermo Fisher Scientific (Pittsburgh, PA, USA). Parallel Artificial Membrane Permeability Assay (Gentest® pre-coated PAMPA plate system) was purchased from Corning Incorporated (Tewksbury, MA, USA).

Synthesis and characterization of β-lactam-based GUMBOS: Synthesis and characterization of octenidine/carbenicillin ([OCT][CAR]) GUMBOS were performed using methods similar to those previously reported by Cole et al. (2015)²³ with slight modification. In the study reported here, octenidine/carbenicillin was easily synthesized using ion-exchange procedures that involved stirring stoichiometric amounts of octenidine dihydrochloride (OCT 2HCl) and disodium carbenicillin (Na₂ CAR) for 1 h at room temperature in deionized water (FIG. 1). The resulting precipitate was washed several times with cold, deionized water and removed using lyophilization overnight. The structure of octenidine/carbenicillin was characterized using ¹Hand ¹³C-NMR and Fourier transform infrared spectroscopy (FT-IR). High-resolution mass spectrometry m/z is not reported as no useful spectrum was obtained, which can sometimes occur with carbenicillin.

Predictive intestinal permeability: PAMPA was employed as an in vitro model of passive, transcellular permeation. In this technique, a 96-well microtiter plate is used as a donor plate and a membrane/acceptor compartment coated with structured tri-layers of phospholipids is placed on top; this configuration is referred to as ‘sandwiched’. Known concentrations of octenidine/carbenicillin (100 μM in 1×PBS, 0.25% DMSO) were added to the donor plate while only buffer was placed in the acceptor plate. The assay was incubated for 5 h at room temperature and the acceptor plate was measured using a UV/Vis spectroscopy plate reader (Eppendorf PlateReader AF2200). Permeability coefficients (Pe) were calculated based on initial concentration in donor well (C₀), concentration in donor well at 5 h (C_{D}), concentration in acceptor well at 5 h (C_{A}), volumes of donor (V_{D}) and acceptor wells (V_{A}), well filter area (A, 0.3 cm²) and incubation time (t, 18000 s), as calculated using the relationship in Equation 1.

$$Pe ( cm / s ) = \frac{- ln [ \frac{{C}_{A}}{\frac{( {C}_{D} \times {C}_{A} ) + [ {C}_{A} \times {V}_{A} ]}{{V}_{D} + {V}_{A}}} ]}{A \times ( \frac{1}{{V}_{D}} + \frac{1}{{V}_{A}} ) \times t} \qquad ( 1 )$$

Characterization of octenidine/carbenicillin GUMBOS: Off-white solid, yield 90%. ¹H-NMR (400 Hz, DMSO-d6) d 9.03 (br s, 1H), 8.64 (d, J=8 Hz, 1H), 8.26 (dd, J=8 Hz, 2H), 8.09 (dd, J=8 Hz, 2H), 7.30-7.19 (m, 6H), 6.95 (dd, J=8 Hz, 2H), 6.89 (dd, J=8Hz, 2H), 5.29-5.25 (m, 2H), 4.08 (t, J=8Hz, 4H), 3.81 (s, 1H), 3.53 (q, 2H), 3.24 (t, J=4 Hz, 8 Hz, 4H), 1.71 (q, 4H), 1.55-1.51 (m, 7H), 1.43 (s, 3H), 1.32-1.22 (m, 31H), 0.85 (t, J=8 Hz, 6H). ¹³C-NMR (125 Hz, DMSO-d6) 170.45, 170.12, 169.23, 156.20, 142.89, 140.34, 135.58, 128.82, 128.72, 128.53, 127.62, 127.39, 125.71, 110.23, 104.33, 77.50, 70.77, 57.45, 56.05, 55.12, 42.43, 41.72, 30.72, 29.69, 28.72, 28.22, 28.19, 28.13, 28.11, 27.84, 27.40, 25.85, 24.82, 21.56, 13.41.

Predictive permeability: Octenidine 2HCl is a dicationic molecule with two chloride anions and this structure does not permeate through skin, mucous membranes, wounds or the placental barrier.³² With an anion exchange metathesis from chloride ions to carbenicillin (a dianion), permeability increased significantly, which inevitably also increased bioavailability. Mean effective Pe for octenidine/carbenicillin was 3.78×10⁻⁶ (±0.85) cm/s, which falls in the range of high permeability. Pe values that are greater than 1.5% 10 cm/s are defined as high permeability, whereas coefficients less than 1.5×10⁻⁶ cm/s are defined as low permeability. This suggests that octenidine carbenicillin and octenidine dihydrochloride behave differently chemically and thus GUMBOS may behave as a new ion pair when used therapeutically.

Antimicrobial susceptibility testing: Kirby-Bauer disc diffusion susceptibility tests were used to determine zones of inhibition (ZOIs) for N. gonorrhoeae (ATCC 49226) and three clinical isolates obtained from Louisiana State University Health Sciences Center New Orleans HIV Outpatient Clinic. Kirby-Bauer disc diffusion is one of the CDC's preferred methods of testing the susceptibility of N. gonorrhoeae. Testing was performed according to CLSI recommended procedures. In this susceptibility test, 6 mm diameter blank paper discs were impregnated with known quantities of antimicrobial drug followed by evaporation of solvent; however, ceftriaxone, azithromycin and doxycycline antimicrobial susceptibility discs were purchased from Thermo Fisher Scientific. Impregnated discs were placed onto prepared nutrient agar plates that were inoculated with N. gonorrhoeae to give a confluent lawn of growth. Suspensions of these various strains were prepared in accordance with a 1.0 McFarland standard. Inoculated agar plates were incubated for 20-24 h at 37° C. in a 5% CO₂ atmosphere.

Cytotoxicity assay: To determine cell viability, a colorimetric MTT dye assay (Promega Corp., Madison, WI, USA) was used, employing the manufacturer's instructions, as an indicator of cytotoxicity of GUMBOS towards healthy (HeLa) cells. Hela cells (ATCC CCL-2) grown in DMEM-reduced serum supplemented with 10% FBS were plated at a density of 1% 10⁵ cells/mL (10⁴ cells/well) in 96-well plates. Concentrations of therapeutic agents up to 500 μM (1% MeOH or DMSO) were doubly diluted in cell culture media and transferred to seeded cells. Cells were incubated for 15 min and 60 min at 37° C. in a 5% CO₂ atmosphere. Cells treated with medium alone served as a negative control. At the end of the incubation period, 15 μL of MTT was added to each well and incubation continued for another hour. Absorbance was measured at 570 nm in a microplate spectrophotometer. All experiments were performed in quadruplicate. Cell viability as a percentage was determined as the ratio between treated cells and untreated (control) cells (taken as 100%). Reported values are the lethal concentrations able to kill 50% of the population of viable cells (IC₅₀).

Antibacterial activity of octenidine/carbenicillin GUMBOS using disc diffusion: Antibacterial activity of octenidine/carbenicillin was compared with that of the individual parent compounds, unreacted stoichiometric mixtures and current therapeutic agents, ceftriaxone, azithromycin and doxycycline, using Kirby-Bauer disc diffusion (Table 3).

TABLE 3
Zones of inhibition for N. gonorrhoeae
ATCC 49226 and clinical isolates

| | Quantity of | Zone size, mm (±SD) | |
|---|---|---|---|
| Test materials | material (nmol) | ATCC 49226 | clinical isolates |
| Ceftriaxone | 50 | 51 ± 1 | 49 ± 4 |
| Azithromycin | 20 | 40 ± 1 | 36 ± 4 |
| Doxycycline | 70 | 35 ± 0.6 | 28 ± 7 |
| [OCT][CAR] | 50 | 40 ± 0.6 | 37 ± 4 |
| 1:1 OCT:CAR | 50 | 30 ± 1 | 34 ± 3 |
| OCT 2HCl | 50 | 9 ± 1 | 9 ± 0.3 |
| Na₂ CAR | 50 | 27 ± 3 | 28 ± 3 |

Blank discs were loaded with 50 nmol of GUMBOS, respective constituent compounds and equivalent stoichiometric unreacted mixtures of octenidine and carbenicillin that are equimolar to the purchased ceftriaxone discs (Oxoid™, Thermo Fisher). After 20-24 h incubation, diameters of ZOIs were measured using a ruler. Ceftriaxone (30 μg, 50 nmol), azithromycin (15 μg, 20 nmol) and doxycycline (30 μg, 70 nmol) had zone sizes within the susceptibility range set by CLSI for N. gonorrhoeae (ATCC 49226) and three clinical isolates tested. Antibacterial activity was improved for octenidine when chloride ions were exchanged for the antibiotic. Through synthesis of octenidine/carbenicillin GUMBOS, antibacterial activity exhibited an additive effect for N. gonorrhoeae (ATCC 49226) and clinical isolates. This effect was not seen, however, for an unreacted mixture of the two drugs for either N. gonorrhoeae (ATCC 49226) or clinical isolates. The ZOIs of octenidine/carbenicillin were also larger than or equal to azithromycin and doxycycline ZOIs for N. gonorrhoeae (ATCC 49226). When comparing ZOIs of octenidine/carbenicillin with those of azithromycin and doxycycline for the clinical isolate population, octenidine/carbenicillin exhibited equal efficacy to azithromycin while exhibiting superior activity to doxycycline, an antibiotic prescribed in the case of gonorrhea and chlamydia coinfection. As a result of these efficacy values, the antibacterial activity of octenidine/carbenicillin was shown to be bioequivalent to azithromycin. This is significant as resistance rates for azithromycin have increased worldwide. Among 57 countries reporting on azithromycin susceptibility, 28 (49%) reported>5% resistance. In the USA, where clinical isolates were obtained, resistance increased significantly from 2014 to 2019; the percentage of isolates with elevated resistance rates to azithromycin increased from 2.5% to 4.6%. Ceftriaxone produced large ZOIs as the clinical population still remains susceptible to it in the USA (only 0.2% of isolates had elevated resistance to ceftriaxone in 2017).

Cytotoxicity of octenidine and carbenicillin in combination and as GUMBOS: Following characterization and antimicrobial efficacy testing, GUMBOS were employed in vitro to assess relative cytotoxicity as compared with the stoichiometric, unreacted mixture and octenidine dihydrochloride. When tested in vitro, β-lactam antibiotics are known to be highly non-toxic. Octenidine dihydrochloride has also shown low cytotoxic potential when tested against human primary gingival fibroblasts and human primary nasal epithelial cells. Octenidine has also been shown to be an effective mouth rinse for substantially reducing oral bacterial counts. For a general approximation of systemic toxicity and possible application beyond mouthwash, such as a vaginal douche, cervical cells were used. Cytotoxicity was assayed at 15 and 60 min because oral rinses are most beneficial if an individual does not drink liquids at least 15 to 60 min after use. If also used to eradicate infections of the cervix, this time frame could allow ample opportunity for the drug to remain in contact with cervical tissue before being removed from the body. The IC₅₀ values for OCT 2HCl, unreacted mixture and octenidine/carbenicillin are reported in Table 4.

TABLE 4
Acute cytotoxicity (IC₅₀) of octenidine/carbenicillin
GUMBOS for 15 and 60 min against HeLa cells

| | IC₅₀ (mg/L) | |
|---|---|---|
| Test materials | 15 min | 60 min |
| OCT 2HCl | 37.4 ± 2.8 | 16.1 ± 0.7 |
| 1:1 OCT:CAR | 52.8 ± 4.8 | 26.6 ± 0.9 |
| [OCT][CAR] | 44.1 ± 2.5 | 23.1 ± 0.2 |

After 15 min of incubation, octenidine/carbenicillin showed lower toxicity than the stoichiometric, unreacted mixture; however, OCT 2HCl was the most toxic to cervical cells. After 60 min of incubation, the toxicity of the unreacted mixture of OCT 2HCl and Na₂ CAR was still lower than that of GUMBOS. While cytotoxicity potential varied between GUMBOS and the parent compound mixture, the unreacted mixture of the two antimicrobial compounds still contains the two sodium ions, which may aggravate hypertension or congestive heart failure.^{41,42} GUMBOS would, therefore, be inherently safer as the sodium ions are removed entirely and replaced with octenidine.

Octenidine/carbenicillin GUMBOS to reduce transmission of gonorrhoea: In 2019, a new paradigm for the transmission of extragenital N. gonorrhoeae emerged. Oropharyngeal infections were noted in the absence of urogenital infections and it was hypothesized that kissing or saliva exchange could be an unrecognized means of transmission. It was proposed that antiseptic mouthwashes might offer a condom-free control strategy. It has been reported that use of Listerine® mouthwash as a gargle could significantly reduce the amount of N. gonorrhoeae in the oropharynx of MSM. It has now been shown that octenidine/carbenicillin GUMBOS could be incorporated into a mouthwash for this purpose.

The constituent components of our GUMBOS, octenidine and carbenicillin, have established safety profiles. Octenidine is currently formulated as a mouthwash and carbenicillin, like most beta-lactam antibiotics, is relatively non-toxic to mammalian cells, as shown in Table 2. The GUMBOS of the present composition can also be advantageously formulated as pessaries or suppositories for use to reduce colonization in other anatomical sites such as the vagina and rectum.

1-34. (canceled)

35. A composition comprising an antiseptic in ionic association with an antibiotic as an ion-pair solid-phase organic salt, wherein the antiseptic and the antibiotic synergistically interact whereby the composition has a Minimal Inhibitory Concentration (MIC) against a sensitive bacterial species that is less than the sum of the MICs of the antiseptic and the antibiotic individually.

36. The composition of claim 35, wherein the antiseptic is effective in modulating the proliferation or viability of a gram-negative strain of a bacterial species when in contact with said strain.

37. The composition of claim 35, wherein the antibiotic is effective in modulating the proliferation or viability of a carbapenem-resistant bacterial strain.

38. The composition of claim 35, wherein the antiseptic is chlorhexidine or octenidine.

39. The composition of claim 35, wherein the antibiotic is a □-lactam.

40. The composition of claim 35, wherein the composition comprises octenidine and ceftriaxone, chlorhexidine and ceftriaxone, or octenidine and carbenicillin.

41. The composition of claim 35, wherein the composition further comprises a pharmaceutical carrier.

42. The composition of claim 41, wherein the composition is formulated for delivery to a subject human or animal intravascularly, or directly to a tissue of the subject.

43. A method of generating a group of uniform materials based on organic salts (GUMBOs) comprising mixing a salt of an antibiotic and a salt of either chlorhexidine or octenidine in water for an extended period, thereby generating an antiseptic-antibiotic ion-pair solid-phase organic salt.

44. The method of claim 43, wherein the chlorhexidine salt is chlorhexidine diacetate salt.

45. The method of claim 43, wherein the antibiotic is a □-lactam.

46. A method of reducing the proliferation or viability of a bacterial strain comprising contacting a population of the bacterial strain or strains with an antiseptic-antibiotic ion-pair solid-phase organic salt.

47. The method of claim 46, wherein the antiseptic is effective in modulating the proliferation or viability of a strain of Neisseria gonorrhoeae or a carbapenem-resistant Enterobacteriaceae.

48. The method of claim 46, wherein the composition comprises at least one of octenidine and ceftriaxone, chlorhexidine and ceftriaxone, and octenidine and carbenicillin.
